(19) United States
(12) Patent Application Publication
(10) Pub. No.: US 2015/0025641 A1
(43) Pub. Date: Jan. 22, 2015

(54) SHOCK-ABSORBING HUMERAL IMPLANT FOR SHOULDER ARTHROPLASTY

(71) Applicant: Marcos V. MASSON, Houston, TX (US)

(72) Inventor: Marcos V. MASSON, Houston, TX (US)

(21) Appl. No.: 14/082,855

(22) Filed: Nov. 18, 2013

Related U.S. Application Data
(60) Provisional application No. 61/727,399, filed on Nov. 16, 2012.
(60) Provisional application No. 61/751,349, filed on Jan. 11, 2013.

(51) Int. Cl. A61F 2/40

(52) U.S. Cl. CPC A61F 2/4081; A61F 2/4059
USPC 623/19.13; 623/19.14

(57) ABSTRACT

A shock-absorbing shoulder replacement system has a glenoid cup suitable for affixing to the glenoid surface of a scapula, ahead positioned so as to bear against the glenoid cup, a rod affixed to or integral with the head on the side of the head opposite the glenoid cup, a humeral component having a shaft suitable for receipt by the humerus and having a portion extending outwardly of the humerus, and a resilient member cooperative between humeral component and the bead such that the head is resiliently mounted in relation to the humeral component. The portion of the humeral component has a receptacle therein suitable for receiving an end of the rod therein. The rod has a threaded surface that can be connected to a nut such that a distance between the head and the humeral component can be adjusted.

### RELATED U.S. APPLICATIONS

The present application claims priority from U.S. Provisional Application No. 61/727,399, filed on Nov. 16, 2012, and entitled “Shock-Absorbing Implant for Use in Reverse Shoulder Arthroscopy”. The present invention also claims priority from U.S. Provisional Patent Application Ser. No. 61/751,349, filed on Jan. 11, 2013, and entitled “Shock-Absorbing Implant with Pressure Adjustment and Sensing for Reverse Shoulder Arthroscopy”.

### STATEMENT REGARDING FEDERALLY SPONSORED RESEARCH OR DEVELOPMENT

Not applicable.

### REFERENCE TO MICROFICHE APPENDIX

Not applicable

### BACKGROUND OF THE INVENTION

1. Field of the Invention

The present invention relates to shoulder replacement surgery. More particularly the present invention the relates to implants that are used in shoulder arthoscopy. More particularly the present invention the relates to a shock-absorbing element incorporated into such implants so as to avoid the risk of fracture and damage to either the surrounding bone and/or implant.

2. Description of Related Art including Information Disclosed Under 37 FR 1.97 and 37 CFR 1.98.

Every year, thousands of conventional total shoulder replacements are successfully carried out in the United States for patients with shoulder arthritis This type of surgery, however, is not as beneficial for patients with large rotator cuff tears who have developed a complex type of shoulder arthritis called “cuff tear arthropathy”. For these patients, conventional total shoulder replacement may result in pain and limited motion, and reverse total shoulder replacement may be an option.

A conventional shoulder replacement device mimics the normal anatomy of the shoulder. In other words, a plastic cup is fitted into the shoulder socket (glenoid), and a metal ball is attached to the top of the humerus. In a reverse total shoulder replacement, the socket and metal ball are switched. The metal ball is fixed to the socket and the plastic cup is fixed to the upper end of the humerus.

A reverse total shoulder replacement works better for people with cuff tear arthropathy because it relies on different muscles to move the arm. In a healthy shoulder, the rotator cuff muscles help position and power the arm during the range of motion. A conventional replacement device also uses the rotator cuff muscles to function properly. In a patient with a large rotator cuff tear and cuff tear arthropathy, these muscles are no longer functional. The reverse total shoulder replacement relies on the deltoid muscle, instead of the rotator cuff, to power and position the arm.

Subsequent to the shoulder replacement surgery, the implant can be subjected to a large variety of forces. In particular, if a patient should fall, the implant may be subjected to extremely large impact forces. These impact forces have been known to fracture the ball which is attached to the glenoid. In other circumstances, the scapula can become fractured because of an impact. In other circumstances, the connection between the socket and the humerus can become loosened or detached. In still other circumstances, the humeral shaft can become fractured as a result of these forces.

Whenever these forces cause a damage to either the implant or to the bone structure, additional surgery may be necessary. Since the ball is attached to the glenoid, it becomes quite difficult to repair and replace the ball after installation. As a result, the implant can become loosened or nonfunctional. In particular, in shoulder replacement surgery, repairs to the implant, following the surgery, are quite complex. As such, a need has developed so as to provide a shoulder implant structure which avoids the damage that can occur from sharp impact forces imparted to the implant.

Shock-absorbing structures have been known to be used in hip replacement surgery. In particular, a variety of patents have issued relating to such shock-absorbing structures. In particular, U.S. Pat. No. 5,201,881, issued on Apr. 13, 1993 to D. L. Evans, shows a joint prosthesis that provides articulating prosthesis components that can deflect with respect to one another so that shock absorption is provided. This serves to lower impact stresses. The components interface at articulating surfaces. A gap is provided at a position away from the articulating surfaces. As a result, one of the components can flex into the gap area during use.

U.S. Pat. No. 5,389,107, issued on Feb. 14, 1995 to Nassar et al., provides a shock absorbent prosthetic hip implant that includes a socket section that is attachable to the pelvic bone and a ball section that is pivotably engaged with the socket section. There is a first shock absorber section attached to the ball section and a second shock absorber section attached to an upper part of the femur bone. The second shock absorber section slidably engages the first shock absorber section. A spring is disposed between the first and second shock absorber sections for cushioning a compressive force applied between the femur and pelvic bones.

U.S. Pat. No. 6,336,941, issued on Jan. 8, 2002 to Subba Rao et al., discloses a modular hip implant with a shock absorption system. The shock absorption system absorbs compressive stresses that are imparted to the implant. A unique coupling member houses a modular spring mechanism that serves as t le shock absorber.

U.S. Pat. No 8,070,823, issued on Dec. 6, 2011 to Kellar et al., teaches a prosthetic ball-and-socket joint. This ball-and-socket joint includes first member having a balanced centroidal axis and includes a rigid material with a concave interior defining a cup surface. The cup surface includes a cantilevered first flange defining a wear-resistant protruding first contact rim. The first flange is asymmetric relative to the balanced centroidal axis. A cantilevered second flange defines a wear-resistant protruding second contact rim. The second member is made of a rigid material with a wear-resistant convex contact surface. The first and second contact rims bear against the contact surface of the second member to transfer loads between the first and second members while allowing pivoting motion therebetween. The flanges are shaped and sized to deform elastically and permit the first and second contact rims to conform in an irregular shape to the contact surface when the joint is under load.

U.S. Patent Publication No. 2002/0143402, published on Oct. 3, 2002 to A. Steinberg, shows a hip joint prostheses that has at least a first and as second load-carrying member. The first load-carrying member is substantially more shock absorbing and resilient than the second load-carrying member.

U.S. Patent Publication No. 2006/0064169, published on Mar. 23, 2006 to B. A. Ferree, discloses shock-absorbing joint and spine replacements. In particular, the joint is a total knee replacement. This total knee replacement implant includes a femoral component having a wheel. The implant has a tibial component that includes a shock-absorbing component with a piston assembly and spring. The implants contain a cushioning or shock-absorbing member to dampen axial loads and other forces. Fluid is forced rapidly from the device wherein compression and dampening is achieved by valves or other pathways that allow for a slower return of the fluid back into the implant as the pressure is relieved.

It is quite natural that shock-absorbing systems have been utilized for knee and hip replacements since the knee and the hip are often subjected to large impact forces. However, typically, with shoulder replacements, the shoulders are not subjected to such strong forces, unless the patient should fall or be involved in an accident. As such, in the past, shock-absorbing systems for shoulder replacement implants have not been developed. As a result, under those circumstances where a patient should fall or be involved in an accident, extensive surgery can be necessary so as to repair the implant and/or the surrounding bone structures. As such, a need has developed so as to provide a shock-absorbing implant for shoulder replacement surgery.

It is an object of the present invention to provide a shock-absorbing implant which minimizes the transmission of forces to the glenoid or to the implant that is attached to the glenoid.

It is another object of the present invention to provide a shock-absorbing implant which mitigates micro-forces.

It is a further object of the present invention to provide a shock-absorbing implant which creates a “weak link” such that an easily repairable portion of the implant will fail prior to a less easily repaired component of the implant.

It is still a further object of the present invention to provide a shock-absorbing implant which avoids any fracturing of the bone and/or damage to the implant.

It is still another object of the present invention to provide a shock-absorbing implant which is easily replaceable.

These and other objects and advantages of the present invention will become apparent from a reading of the attached specification and appended claims.

### BRIEF SUMMARY OF THE INVENTION

The present invention is a shock-absorbing shoulder replacement system that includes a glenoid cup suitable for fixing to the glenoid surface of a scapula, a head positioned so as to bear against the glenoid cup, a rod affixed to or integral with the head on the side of the head opposite the glenoid cup, a humeral component having a shaft suitable for receipt by the humerus and having a portion extending outwardly of the humerus, and a resilient member cooperative between humeral component and the head such that the head is resiliently mounted in relation to the humeral component. The humeral component has a receptacle suitable for receiving the rod therein.

The rod has a threaded surface. A nut is threadedly positioned over the threaded surface of the rod. A housing serves to receive the nut therein. The housing has a bole formed therein. The rod extends through the hole such that the nut is positioned on the threaded surface within the housing.

The resilient member has an end affixed to the housing and opposite end affixed to the humeral component. The resilient member can be a compression spring.

The portion of humeral component has a receptacle formed therein. The rod has an end opposite the head positioned in this receptacle. The receptacle has a hole opening there into. The bole has a diameter. The rod has a flange formed adjacent the end opposite the head. This flange has a diameter greater than the diameter of the bole. As such, the rod is slidable in relation to the hole such that the end of the rod can move forwardly in rearwardly in the receptacle. The walls of the receptacle serve as stops or limits to the length of travel between the bead and the humeral component.

The head has a generally semi-spherical shape. The glenoid cup includes a base suitable for affixing to the glenoid surface and a bearing surface affixed to the base. The head bears against the bearing surface. The base has a plurality of pegs extending outwardly of the side of the base opposite the bearing surface. The pegs are suitable for engaging the glenoid surface. The base has a rim extending therearound. The bearing surface is retained within the rim.

The present invention is also a shock-absorbing humeral implant that includes a head, a rod having one end affixed to or integral with the head, a humeral Component has a receptacle suitable for receiving and opposite end of the rod therein, and a resilient member cooperative the humeral component and the head such that the head is resiliently mounted in relation to the humeral component.

In the shock-absorbing humeral implant, a housing is affixed to the resilient member opposite the humeral component. The rod extends through the housing. A nut is fixedly positioned in the housing. The nut is suitable for threadedly and adjustably engaging the threaded surface of the rod. The head is rotatable with respect to the housing such that a rotation of the head causes a distance between the head and humeral component to increase or decrease.

The outwardly extending portion of humeral component has a receptacle formed therein. The rod has an end opposite the head that is positioned in the receptacle. The receptacle as a hole opening thereinto. The hole has a diameter. The rod has a flange adjacent the end opposite the head. This flange has a diameter greater than the diameter of the hole. The rod is slidable in relation to the hole such that the end of the rod opposite the head can move forwardly and rearwardly in the receptacle.

This foregoing Section is intended to describe, with particularity, the preferred embodiment of the present invention. It is understood that modifications to this preferred embodiment can be made within the scope of the present invention. As such, this Section should not to be construed, in any way, as limiting of the scope of the present invention. The present invention should only be limited by the following claims and their legal equivalents.

### BRIEF DESCRIPTION OF THE SEVERAL VIEWS OF THE DRAWINGS

FIG. 1 is an illustration in side elevational view showing the shock-absorbing shoulder replacement system and shock-absorbing humeral implant in accordance with the teachings of the present invention.

### DETAILED DESCRIPTION OF THE INVENTION

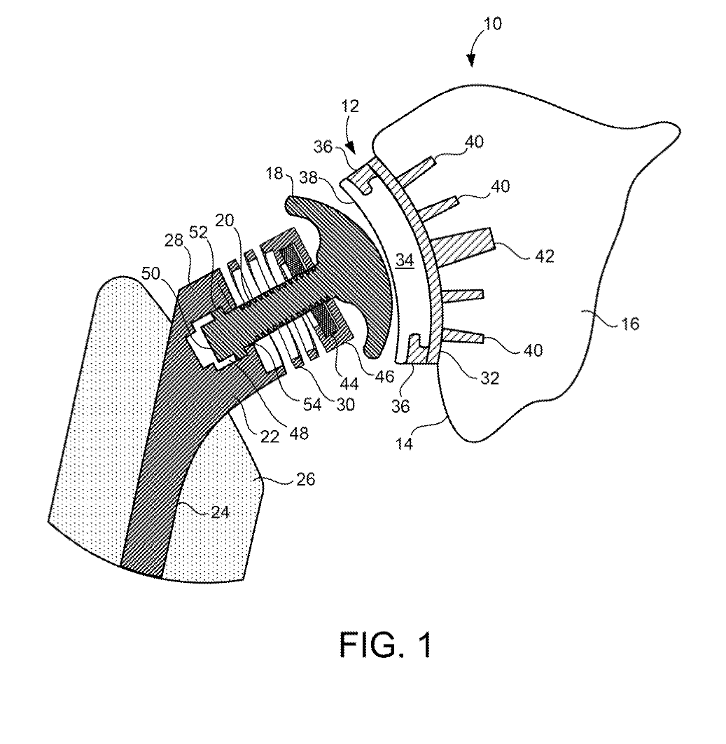

Referring to FIG. 1, there is shown the shock-absorbing shoulder replacement system 10 in accordance with the teachings of the present invention. The shock-absorbing shoulder replacement system 10 includes a glenoid cup 12 suitable for fixing to the glenoid surface 14 of a scapula 16. A head 18 is positioned so as to bear against the glenoid cup 12. A rod 20 is affixed to or integral with the head 18 on a side of the head 18 opposite the glenoid cup 12. There is a humeral component 22 that has a shaft 24 suitable for receipt by the humerus 26. The humeral component 22 has a portion 28 at an end thereof. Portion 28 is suitable for extending outwardly of the humerus 26 when the shaft 28 is implanted therein. A resilient member 30 is cooperative between the humeral component 22 and the head 18 such that the head is resiliently mounted in relation to the humeral component 22.

In FIG. 1, can be seen that the glenoid cup 12 has a base 32 that is suitable for fixing to the glenoid surface 14. The glenoid cup 12 also includes a bearing surface 34 that is affixed to the base 32. The head 18 will bear against the beating surface. The bearing surface can be of a polymeric material, such as polyethylene. It can be seen that the base 32 has a rim 36 extending therearound. The bearing surface 34 is retained within the rim 36. The bearing surface 34 has an outer surface 38 of a concave shape. As such, the convex head 18 can suitably bear against and move in relation to the outer surface 38 of the bearing surface 34.

The base 32 has a plurality of pegs 40 extending from an inner surface thereof. Pegs 40 are suitable for engaging with the glenoid surface 14 for creating the implant. A screw 42 can be inserted through a hole in the base 32 so as to affix the base 32 and the bearing surface 34 in a proper position on the glenoid surface 14.

The head 18 has a generally semi-spherical shape. The rod 20 is either affixed to or integral with the head 18 on the side of the head 18 opposite the bearing surface 34. It can be seen that the rod 30 has an exterior thread formed thereon.

Importantly, there is a nut 44 that is threadedly secured to the threaded exterior surface of the rod 20. The nut 44 is retained within a housing 46. As such, the nut 44 will have a fixed position relative to the rotatable head 18. As result, if adjustments in the distance between the head 18 and the humeral component 22 are necessary, then the head 18 can be suitably adjusted by rotation. A rotation of the head 18 in one direction will tend to draw the head 18 toward the portion 28 of the humeral component 22. A rotation in the opposite direction will tend to cause the head 18 to move away from the portion 28 of the humeral component 22. The limit of travel of the head 18 cart be suitably governed by the inner walls of a receptacle 48 formed in the portion 28 of the humeral component 22.

In particular, the shaft 20 has an end 50 that is received within the receptacle 48. As such, the end 50 (opposite the head 18) can move forwardly or rearwardly within the receptacle 18. The rod 20 has a flange 52 extending Gum outwardly therefrom. The rod 52 that extends through a hole 54 formed adjacent to the receptacle 48 in the portion 28 of the humeral component 22. The flange 52 will have a diameter greater than the diameter of the hole 54. As such, the end 50 of the rod 20 is securely maintained within the receptacle 48.

The resilient member 30 is a compression spring. One end of the compression spring is connected to the portion 28 of the humeral component 24. An opposite end of the resilient member 30 is affixed to the housing 46. As result, the housing 46, along with the nut 44, will engage with the rod 20 so as to create the resilient mounting effect for the head 18. It can be seen that the bead the 18 has the rod 20 extending through holes in the housing 46 such that the rod can extend through the interior of the resilient member 30.

Through the use of the implant system 10 of the present invention, when a patient should fall or be involved in an accident, extreme forces can be imparted to the implant system 10. lithe forces are at a certain level, the spring 30 will compress so as to avoid peak loads on the system. Ultimately, the implant system can return to a home position. As result, these peak loads are effectively absorbed and are not transmitted to the humeral component 22 or to the glenoid cup 12. As a result, the damage and fracturing of the head 18 of the implant is avoided. In other circumstances, and more extreme forces are involved, it is possible for the rod 22 break. As such, the rod 22 can act as a “weak link”. As result, relatively simple surgery can be carried out so as to replace the damaged component. As such, the system 10 will sacrifice itself so as to avoid damage to the glenoid cup 12 and/or to the humeral component 22. The result of the present invention is that the present invention effectively prevents damage to the shoulder replacement system and avoids complicated surgical procedures that may be necessary so as to either repair the glenoid cup 12, the glenoid implant, the humeral component 22 or the humeral implant.

The foregoing disclosure and description of the invention is illustrative and explanatory thereof. Various changes in the details of the illustrated construction can be made within the scope of the present invention without departing from the true spirit of the present invention. The present invention should only be limited by the following claims and their legal equivalents.

I claim:

1. A shock-absorbing shoulder replacement system comprising:
glenoid cup suitable for affixing to a glenoid surface of a scapula;
a bead positioned so as to bear against said glenoid cup;
a rod affixed to or integral with said head on a side of said head opposite said glenoid cup;
a humeral component having a shaft suitable for receipt by the humerus, said humeral component having a portion extending outwardly of the humerus, said portion receiving said rod therein; and
a resilient member cooperative between said humeral component and said bead such that said head is resiliently mounted in relation to said humeral component.

2. The shock-absorbing shoulder replacement system of claim 1, said rod having a threaded surface, the shock-absorbing shoulder replacement system further comprising:
a nut threadedly positioned on said threaded surface of said rod.

3. The shock-absorbing shoulder replacement system of claim 2, further comprising:
a housing receiving said nut therein, said housing having a hole formed therein, said rod extending to said hole such that said nut is positioned on said threaded surface within said housing.

4. The shock-absorbing shoulder replacement system of claim 3, said resilient member having an end affixed to said housing and an opposite end affixed to said humeral component.

5. The shock-absorbing shoulder replacement system of Claim said portion of said humeral component having a receptacle formed therein, said rod haying an end opposite said bead positioned in said receptacle.

6. The shock-absorbing shoulder replacement system of claim 5, said receptacle haying a hole opening thereinto, said bole having a diameter, said rod having a flange adjacent said end opposite said head, said flange having a diameter greater than said diameter of said hole.

7. The shock-absorbing shoulder replacement system of claim 6, said rod being slidable in relation to said hole such that said end of said rod opposite said hole can move forwardly and rearwardly in said receptacle.

8. The shock-absorbing shoulder replacement system of claim 1, said head haying a generally semi-spherical shape.

9. The shock-absorbing shoulder replacement system of claim 1, said glenoid cup comprising:
a base suitable for fixing to the glenoid surface; and
a bearing surface affixed to said base, said bead bearing against said bearing surface.

10. The shock-absorbing shoulder replacement system of claim 9, said base having a plurality of pegs extending outwardly of the side of said base opposite said bearing surface, said peg suitable for engaging the glenoid surface, said base baying a rim extending therearound, said bearing surface retained within said rim.

11. A shock-absorbing humeral implant comprising:
a bead;
a rod having one end affixed to or integral with said head;
a humeral component basing a portion at an end thereof, said portion having a receptacle receiving an opposite end of said rod therein; and
a resilient member cooperative with said humeral component and said head such that said head is resiliently mounted in relation to said humeral component.

12. The shock-absorbing humeral implant of claim 11, further comprising:
a housing affixed to said resilient member opposite said humeral component, said rod extending through said housing.

13. The shock-absorbing humeral implant of claim 12, further comprising:
a nut positioned in said housing, said rod having a threaded surface, said nut threadedly and adjustably affixed to the threaded surface of said rod.

14. The shock-absorbing humeral implant of claim 12, said head being rotatable with respect to said housing such that a rotation of said head causes a distance between said head and said humeral component to increase or decrease.

15. The shock-absorbing humeral implant of claim 11, said portion of said humeral component having a receptacle formed therein, said rod having an end opposite said head positioned in said receptacle.

16. The shock-absorbing humeral implant of claim 15, said receptacle having a hole opening thereinto, said hole having a diameter, said rod having a flange adjacent said end opposite said head, said flange having a diameter greater than said diameter of said hole.

17. The shock-absorbing humeral implant of claim 16, said rod being slidable in relation to said hole such that said end of said rod opposite said head can move forwardly and rearwardly in said receptacle.

18. A shock-absorbing humeral implant comprising:
a head;
a threaded rod having one end affixed to or integral with said head;
a housing being threadedly connected to said threaded rod;
humeral component; and
a resilient member cooperative with said humeral component and said head such that said head is resiliently mounted in relation to said humeral component.

19. The shock-absorbing humeral implant of claim 18, said housing having a nut therein, said nut been threadedly affixed to said rod.

20. The shock-absorbing humeral implant of claim 18, said resilient member being a compression spring having one end affixed to said housing and an opposite end affixed to said humeral component.
